(19) United States
(12) Patent Application Publication
(10) Pub. No.: US 2025/0025626 A1
(43) Pub. Date: Jan. 23, 2025

(54) INFUSION HUB, INFUSION SET, AND ASSOCIATED METHODS

(71) Applicant: Unomedical A/S, Lejre (DK)

(72) Inventors: Omid Mostafaei, Lejre (DK); Lisa Jaunet van Kooten, Copenhagen (DK)

(21) Appl. No.: 18/902,798

(22) Filed: Sep. 30, 2024

Related U.S. Application Data
(63) Continuation of application No. PCT/EP2023/059065, filed on Apr. 5, 2023.
(60) Provisional application No. 63/328,806, filed on Apr. 8, 2022.
(60) Provisional application No. 63/413,360, filed on Oct. 5, 2022.
(60) Provisional application No. 63/413,370, filed on Oct. 5, 2022.

(30) Foreign Application Priority Data
May 23, 2022 (GB) 2207515.4
Nov. 22, 2022 (GB) 2217425.4
Dec. 7, 2022 (GB) 2218352.9

(51) Int. Cl. A61M 5/142; A61M 5/165

(52) U.S. Cl. CPC A61M 5/14244; A61M 5/165; A61M 2205/75

(57) ABSTRACT

An infusion hub for subcutaneous infusion of a therapeutic agent into a patient, the infusion hub comprising: a casing; a cannula for insertion into a patient; a fluid transfer part connected to the casing and in fluid communication with the cannula; and, a tubing connector configured to engage the fluid transfer part and connectable to tubing for receiving a therapeutic agent from a pump; wherein the cannula defines a downstream end of a fluid flow path extending between a pump and the patient via the fluid transfer part; and, wherein the infusion hub comprises a filter disposed in the tubing connector.

### FIELD

The invention relates to an infusion hub and an infusion set for subcutaneous infusion of a therapeutic agent into a patient, and associated methods. Particularly, though not exclusively, the invention relates to infusion sets for subcutaneous infusion of insulin, heparin, apomorphine, arbidopa, or levodopa and/or levodopa products into a patient.

### BACKGROUND

For patients with diabetes, insulin therapy is often an important part of their treatment, helping to regulate blood sugar levels and store excess glucose for energy. There are two principal modes for delivering insulin. The first mode includes syringes and injector pens, which are used to inject a dose of insulin typically three to four times a day (depending on, inter alia, the type of diabetes and blood sugar levels of the patient). While these devices are simple and low cost, delivering each dose of insulin requires a needle stick. The second mode uses an infusion pump, sometimes called an insulin pump, which delivers controlled doses of insulin throughout the day. An infusion pump can be used to deliver insulin to a patient continuously (basal dose), on demand (bolus dose) or at scheduled intervals. Infusion pumps are more complex and expensive than syringes and pens, though enable improved regulation of blood sugar levels, for example by programmable delivery schedules, and requires fewer needle sticks.

The second mode is known as continuous subcutaneous insulin infusion (CSII) therapy. Infusion pump systems for CSII therapy may be worn by the patient. The systems typically include a combined infusion pump and reservoir fro containing an insulin drug, for example human insulin or analogue insulin, and an insulin infusion set. The infusion set may include a cannula (for example, a polymeric catheter or metal needle) for insertion subcutaneously into the patient and flexible tubing for fluidly connecting the cannula to the reservoir. Once the cannula is inserted into the patient, it may remain in place for a period of time, i.e., days, to allow for continuous delivery of the insulin drug. The current recommended wear time for insulin infusion sets is two to three days, to avoid problems that may arise relating to the infusion set itself or to the infusion site. However, such problems may still arise within recommended wear times, resulting in early removal of the infusion set and more frequent site rotation across infusion sites (for example buttocks, abdomen and arms).

While problems relating to the infusion set have been well investigated and addressed in recent years, there remains little understanding and few solutions to address problems relating to the infusion site. Problems relating to the infusion site include pain, bleeding, infection, skin irritation, erythema, lipohypertrophy and lipoatrophy. Problems at the infusion site may lead to the build-up of scar tissue, which consequently lowers insulin sensitivity and increases the risk of hypoglycaemia, as well as having a cosmetic impact on patients. All these problems can deter patients from continuing to use their infusion pumps, resulting in poorer patient outcomes.

It is known that problems at the infusion site are a consequence of the immune response to the presence of the cannula and the insulin drug in the body. The immune system responds by activating and progressing the foreign body reaction (FBR)—an inflammatory and fibrotic process that occurs upon introducing a foreign material into the body. In FBR, cells of the immune system identify foreign materials (such as unwanted biological, chemical, or physical species, present in infusible solutions) and attempt to degrade it, or otherwise encapsulate the material by forming a physical barrier to isolate it from the rest of the body. FBR is a problem for increasing wear times of infusion sets in CSII therapy, as the immune system reacts to the inserted cannula and the insulin drug. This limitation prevents realising the full potential of CSII therapy.

Generally, unwanted species, whether biological, chemical or physical, present in infusible solutions have undesirable consequences for patients.

It is an object of embodiments of the invention to provide an improved infusion set that attempts to circumvent FBR, increase wear times of infusion sets, and/or at least mitigate one or more problems associated with known arrangements.

### BRIEF SUMMARY OF THE DISCLOSURE

The invention is defined by the appended claims.

The present disclosure provides an infusion hub for subcutaneous infusion of a therapeutic agent into a patient, the infusion hub comprising: a casing; a cannula for insertion into a patient; a fluid transfer part connected to the casing and in fluid communication with the cannula; and, a tubing connector configured to engage the fluid transfer part and connectable to tubing for receiving a therapeutic agent from a pump; wherein the cannula defines a downstream end of a fluid flow path extending between a pump and the patient via the fluid transfer part; and, wherein the infusion hub comprises a filter disposed in the tubing connector.

The present disclosure also provides a method of treating a patient via subcutaneous infusion with a therapeutic agent, the method comprising the steps of: placing an infusion hub as described herein at an infusion site of a patient, connecting, via tubing, a pump fluidly connected to a source of a therapeutic agent to the tubing connector, priming the tubing with the therapeutic agent, and injecting the patient with the therapeutic agent.

The present disclosure also provides a method of treating a patient via subcutaneous infusion with a therapeutic agent, the method comprising the steps of:
providing an infusion hub as described herein, connecting, via tubing, a pump fluidly connected to a source of a therapeutic agent to the tubing connector, priming the tubing with the therapeutic agent, placing the infusion hub at an infusion site of a patient, and injecting the patient with the therapeutic agent, connecting tubing between the tubing connector of the infusion hub and a pump; inserting the cannula into the patient; and, introducing the therapeutic agent into the patient.

The present disclosure also provides an infusion hub for subcutaneous infusion of a therapeutic agent into a patient, the infusion hub comprising: a casing; a cannula for insertion into a patient; a fluid transfer part, connected to the casing and in fluid communication with the cannula; and, a tubing connector configured to engage the fluid transfer part and connectable to tubing for receiving a therapeutic agent from a pump; wherein the cannula defines a downstream end of a fluid flow path extending between a pump and the patient via the fluid transfer part; and, wherein the infusion hub comprises a filter disposed in the fluid flow path within the fluid transfer part.

The present disclosure also provides an infusion set comprising: an infusion hub as described herein; and, tubing connectable between the tubing connector of the infusion hub and a pump.

In some cases, the infusion set further comprises a pump for containing and delivering the therapeutic agent to the infusion hub via the tubing.

In some cases, an interface of the fluid transfer part comprises a sealing membrane configured to prevent egress of fluid out of the fluid transfer part from the fluid flow path.

In some cases, when the tubing connector is secured to the fluid transfer part, a needle of the tubing connector pierces the sealing membrane of the fluid transfer part.

In some cases, the tubing connector comprises an upstream fluid lumen, a downstream fluid lumen, and a cavity provided between the upstream fluid lumen and the downstream fluid lumen; wherein the filter is disposed in the fluid flow path within the cavity.

In some cases, the filter is disposed in the fluid flow path between an interface of the fluid transfer part and the cannula.

In some cases, the filter provides a physical filter medium for removing unwanted species, for example, by size exclusion.

In some cases, the filter provides a chemical filter medium for removing unwanted species, for example, by sorption.

In some cases, the filter is a modular filter comprising first and second sub-filters arranged to allow fluid flow therethrough in series.

In some cases, the first sub-filter comprises a filter material and the second subfilter comprises a further filter material different to the filter material.

In some cases, the filter comprises a foam. The foam may comprise any combination of a cellulose, a polyurethane (Pll), a polyester, a polyether and a collagen. While a foam is described herein, it would be apparent other structures, such as a membrane or a sheet or similar may be used in place of a foam and the properties described in relation to foams apply equally to membranes or sheets.

In some cases, the therapeutic agent comprises insulin, heparin, apomorphine, arbidopa, or levodopa and/or levodopa products.

In some cases, the cannula may comprise a lumen wall having surface features for maintaining and/or locating the filter within the cannula. The cannula may be a soft polymeric catheter or a metal needle. The cannula may comprise one of a polytetrafluoroethylene (PTFE), a fluorinated ethylene propylene (FEP), a rubber, a polyethylene (PE), a polyurethane (Pll), a polypropylene (PP) or a silicone material. The cannula may be insertable in the patient by an insertion needle.

Infusion sets with filters and associated methods as described herein may be useful in inhibiting FBR at an infusion site, and thereby may avoid problematic occurrences such as coagulation, occlusion and/or inflammation at the infusion site, and/or encapsulation of the cannula. In particular, devices and methods as described herein may be useful in inhibiting FBR at the infusion site in diabetic patients receiving CSII therapy. The infusion site may be a single infusion site in use for an extended period of time, for example at least four days.

Infusion sets with filters and associated methods as described herein may be useful in removing unwanted species from a therapeutic agent before delivery of the therapeutic agent to a patient, for example where unwanted species include preservatives necessarily present in insulin solutions to stabilise and/or sterilise insulin solutions prior to delivery to a patient, but which are cytotoxic.

### BRIEF DESCRIPTION OF THE DRAWINGS

Exemplary infusion sets and associated methods are further described hereinafter with reference to the accompanying drawings, in which:

FIG. 1A is an illustration of an infusion set;

FIG. 1B is a further illustration of the infusion set of FIG. 1A;

FIG. 2A is a side cross-section view of an exemplary infusion set;

FIG. 2B is a perspective view of the infusion set of FIG. 2A;

FIG. 2C is a plan cross-section view of the infusion set of FIG. 2A;

FIG. 3A is a side cross-section view of a further exemplary infusion set; and,

FIG. 3B is a plan cross-section view of the infusion set of FIG. 3A.

### DETAILED DESCRIPTION

The presently described infusion sets and associated methods have particular application for use with infusion pump systems such as an infusion pump for delivery of a therapeutic agent, such as insulin, heparin or any other liquid therapeutic agents, where the infusion pump includes a fluid pump and a reservoir, and an infusion set having a cannula (typically part of an infusion hub) and tubing for connecting the cannula to the reservoir. The infusion pump may be an insulin pump for CSII therapy, and the therapeutic agent may be an insulin formulation. The presently described infusion sets and associated methods are able to deliver insulin to a patient at a single infusion site over an extended period of time. An extended period of time is to be understood to mean at least four days. More specifically, an extended period of time may include four to seven days, seven or more days, seven to 10 days, and 10 or more days. An extended period of time may include 14 or more days.

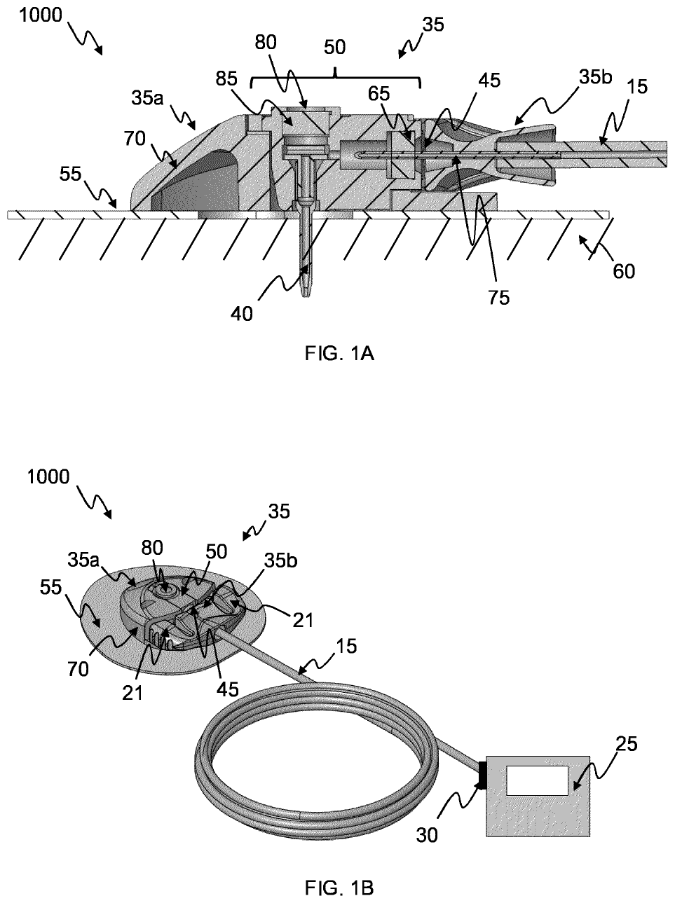

FIGS. 1A and 1B illustrate an infusion set 1000 on an infusion site. The infusion set 1000 includes an infusion hub 35, including a body 35a and a tubing connector 35b. The infusion hub 35 is secured to the skin of a patient by an adhesive patch 55 which maintains a cannula 40 subcutaneously within the sub-dermal fatty tissue of a patient. Tubing 15 connects a pump outlet connector 30 of a pump 25 (the pump 25 containing the therapeutic agent) with the infusion hub 35 via the tubing connector 35b. The body 35a includes a casing 70, and a fluid transfer part 50 which provides a fluid channel. The fluid transfer part is secured within the casing 70 and retains the cannula 40 within the patient. The fluid part 50 may be integral with the casing 70.

The tubing connector 35b engages an interface 45 of the fluid transfer part 50 to connect a downstream end of the tubing 15 to the fluid transfer part 50. The tubing connector 35b includes a releasable connector, in this case a releasable snap-fit joint. As shown in FIG. 1B, the tubing connector 35b has mechanical elements in the form of resiliently deformable arms 21 that engage corresponding mechanical receiving elements on the body 35a of the infusion hub 35, or more specifically, the casing 70 of the body 35a. While the mechanical elements are arranged as a releasable clip in the illustrated tubing connector 35b, it would be apparent that this is not essential. When the tubing connector 35b is secured to the body 35a of the infusion hub 35, a needle 75 of the tubing connector 35b pierces a sealing membrane 65 within the fluid transfer part 50, so that the therapeutic agent can flow through the fluid transfer part 50 and into the patient via the cannula 40.

The function of the fluid transfer part 50 is to allow fluid (e.g. a therapeutic agent) to be transferred through the infusion hub 35 (i.e., from the interface 45 of the fluid transfer part 50 to the cannula 40). As can be seen in FIG. 1A, the fluid transfer part 50 provides a fluid flow path that extends firstly approximately parallel to the plane of the skin surface, and then bends to be approximately perpendicular to the skin surface.

The fluid transfer part 50 has multiple interfaces 45, 80. A sealing membrane 65 seals a first interface 45, and a second sealing membrane 85 is used to seal a second interface 80. While two interfaces 45, 80 are shown, it would be apparent more than two interfaces may be provided as required. Some or all the interfaces may have a sealing membrane secured therein to prevent egress of therapeutic agent from the fluid transfer part 50, through the respective interface.

The cannula 40 is a substantially tubular member for insertion in, and delivering a therapeutic agent to, an infusion site 60. The therapeutic agent includes insulin or an insulin solution. A proximal end of the cannula 40 is fluidly connected to a source of a therapeutic agent, here an infusion pump 25. An opposing, distal end of the cannula 40 is positioned in the infusion site 60, extending to a desired depth to deliver the therapeutic agent. The cannula 40 is any suitable cannula suitable for implantation in a tissue site of a patient, such as a polymeric catheter or metal needle.

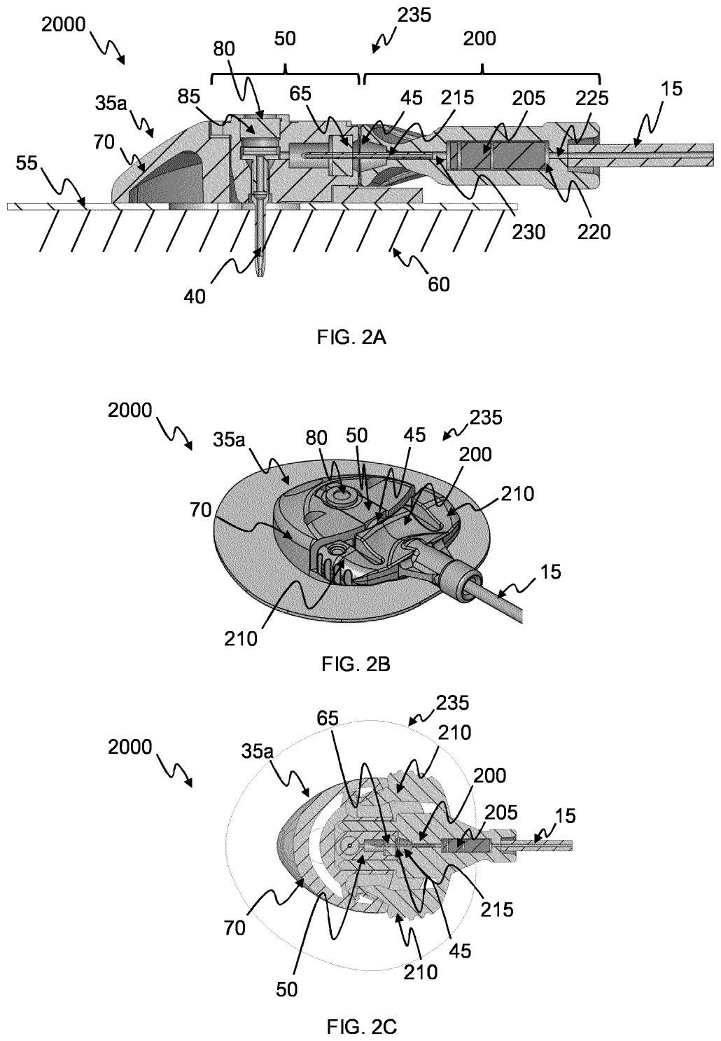

FIGS. 2A to 2C illustrate an exemplary infusion set 2000. Like components described above are denoted by the same reference numerals in FIGS. 2A to 2C. The infusion set 2000 includes an infusion hub 235 including a tubing connector 200 for connecting to the body 35a. The body 35a includes a casing 70, and a fluid transfer part 50 which is secured within the casing 70 and retains the cannula 40 within the patient, as described above. The tubing connector 200 differs from the tubing connector 35b, as described below.

The tubing connector 200 engages an interface 45 of the fluid transfer part 50 to connect a downstream end of the tubing 15 to the fluid transfer part 50. The tubing connector 200 includes a releasable connector, in this case a releasable snap-fit joint. As shown in FIG. 2B and 2C, the tubing connector 200 has mechanical elements in the form of resiliently deformable arms 210 that engage corresponding mechanical receiving elements on the body 35a of the infusion hub 235, or more specifically, on the casing 70 of the body 35a. While the mechanical elements are arranged as a releasable clip in the illustrated tubing connector 200, it would be apparent that this is not essential and in some cases the mechanical elements are not releasable. When the tubing connector 200 is secured to the body 35a, a needle 215 of the tubing connector 200 pierces a sealing membrane 65 within the fluid transfer part 50, so that the therapeutic agent can flow through the fluid transfer part 50 and into the patient via the cannula 40. The sealing membrane 65 prevents therapeutic agent from leaking out of the fluid transfer part 50.

The tubing connector 200 forms part of the fluid flow path between the pump 25 and the patient and includes a filter 205 for removing unwanted species from the therapeutic agent prior to delivery of the therapeutic agent to the patient, close to end of the fluid flow path. The filter 205 is located within the fluid flow path of the tubing connector 200 in a cavity 220 between the inlet and outlet ends of the tubing connector 200. The cavity 220 has a geometry substantially corresponding to that of the filter 205. The filter 225 may be cylindrical, conical, a sheet or any functional form in shape. Further, the filter 205 is sized to fit at least the width of the cavity 220, such that any fluid (e.g., a therapeutic agent) in the fluid flow path necessarily passes through the filter 205. The tubing connector 200 has an upstream fluid lumen 225 that operatively connects the tubing 15 with the cavity 220. At the other end of the cavity 220, a downstream fluid lumen 230 connects the cavity 220 with a needle 215 that penetrates the sealing membrane 65 of the fluid transfer part 50.

The tubing connector 200 is connected, preferably releasably, to the tubing 15. The tubing connector 200 can be connected to the tubing 15 by gluing or welding (e.g., ultrasonic welding) at an upstream end of the tubing connector 200. A person skilled in the art would appreciate that alternate methods of connecting the tubing connector 200 and the tubing 15 are possible.

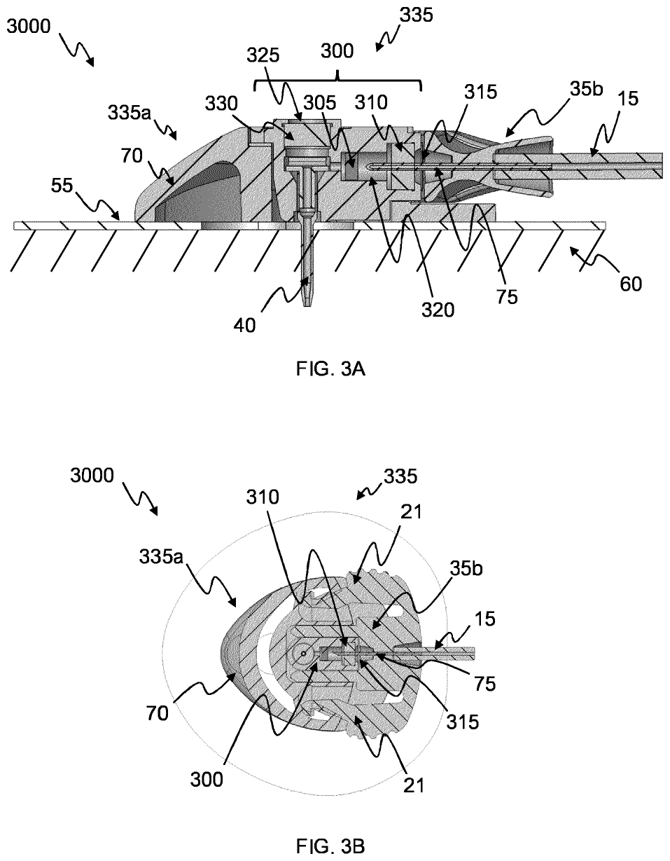

FIGS. 3A and 3B illustrate an exemplary infusion set 3000. Like components described above are denoted by the same reference numerals in FIGS. 3A to 3B. The infusion set 3000 includes an infusion hub 335, including a body 335a and a tubing connector 35b. The body 335a includes a casing 70, and a fluid transfer part 300 which is secured within the casing 70. The fluid transfer part 300 retains the cannula 40 within the infusion hub 335, and provides a fluid connection between the tubing 15 and the cannula 40 of the infusion set 3000. The fluid transfer part 300 may be integral to the casing 70 of the body 335a. Alternatively, the fluid transfer part 300 may be releasably connectable to the casing 70 of the body 335a.

The infusion hub 335 is secured to the skin of a patient by an adhesive patch 55 which maintains a cannula 40 subcutaneously within the sub-dermal fatty tissue of a patient. The adhesive patch 55 extends across the entire underside of the body 335a of the infusion hub 335, to provide good adhesion to the skin.

The tubing 15 is connected to the body 335a of the infusion hub 335 by a tubing connector 35b, which engages an interface 315 of the fluid transfer part 300. As shown in FIG. 3B, the tubing connector 35b has resiliently deformable arms 21 for releasably securing the tubing connector 35b to the body 335a of the infusion hub 335, and more specifically the casing 70 of the body 335a, as explained above. When the tubing connector 35b is secured to the body 335a, a needle 75 of the tubing connector 35b pierces a sealing membrane 310 within the fluid transfer part 300, so that the therapeutic agent can flow through the fluid transfer part 300 and into the patient via the cannula 40. The sealing membrane 310 prevents egress of the therapeutic agent out of the fluid transfer part 300. The fluid transfer part 300 forms part of the fluid flow path, such that the cannula 40 defines a downstream end of a fluid flow path extending between the pump outlet connector 30 and the patient via the fluid transfer part 300.

The fluid transfer part 300 further includes a filter 305 disposed in the fluid flow path within a cavity 320 defined between the interface 315 of the fluid transfer part 300 and the cannula 40. The cavity 320 has a geometry substantially corresponding to that of the filter 305. The filter 325 may be cylindrical, conical, a sheet or any functional form in shape. Further, the filter 305 is sized to fit at least the width of the cavity 320, such that any fluid (e.g., a therapeutic agent) in the fluid flow path necessarily passes through the filter 305. More specifically, the cavity 320 is a cylindrical void in the fluid transfer part 300, located in the fluid flow path after the sealing membrane 310, and at the end of a needle 75 of the tubing connector 35b, connecting from the tubing 15. It would be apparent that the cavity 320 may have any functional shape corresponding to the filter. The needle 75 provides a fluid flow path through the tubing connector 35b, through the sealing membrane 310, and meets the cavity 320.

The function of the fluid transfer part 300 is to allow fluid (e.g. a therapeutic agent) to be transferred through the infusion hub 335 (i.e., from the interface 315 of the fluid transfer part 300 to the cannula 40). As can be seen in FIG. 3A, the fluid transfer part 300 provides a fluid flow path that extends firstly approximately parallel to the plane of the skin surface, and then bends to be approximately perpendicular to the skin surface.

The fluid transfer part 300 has multiple interfaces 315, 325. A sealing membrane 310 seals a first interface 315, and a second sealing membrane 330 is used to seal a second interface 325. While two interfaces 315, 325 are shown, it would be apparent more than two interfaces may be provided as required. Some or all the interfaces may have a sealing membrane secured therein to prevent egress of therapeutic agent from the fluid transfer part 300 through the respective interface.

Locating the filter 305 within the fluid transfer part 300, proximate the cannula 40, allows the filter 305 to remove unwanted species, e.g., preservatives, from the therapeutic agent proximate the point of delivery to the patient, i.e., close to an end of the fluid flow path between the source of the therapeutic agent (the infusion pump 25) and the infusion site 60 thereby, the therapeutic agent is stable throughout the infusion set.

The infusion sets 2000, 3000 described above each include a filter 205, 305. The filter is intended to remove, for example by filtration, unwanted species present in the therapeutic agent to circumvent FBR. Used herein, unwanted species is to be understood to mean one or more species which may be present in the therapeutic agent, for example by design or accident, and which may be undesirable to remain in the therapeutic agent at the point of delivery to the infusion site. In particular, the filter may remove unwanted species that occur in insulin solutions. Such unwanted species may be particulate and/or molecular in nature. Examples of particulate unwanted species include plastic particles, dust and insulin agglomerates, which have been produced during manufacture, storage, sterilization, or handling of the infusion set and/or the insulin solution. Examples of molecular unwanted species include preservatives commonly used in insulin solutions, such as phenol, cresol (particularly m-cresol), benzyl alcohol, benzalkonium chloride, cetrimide, chlorobutanol, chlorhexidine, chlorocresol, hydroxy benzoates, phenethyl alcohol, phenoxyethanol and phenylmercuric nitrate.

The filter may include any filter material capable of removing one or more unwanted species from the therapeutic agent. To remove particulate unwanted species, the filter material may provide a physical filter medium for removing unwanted species by size exclusion, including whereby the filter material functions as a molecular sieve. Additionally, or alternatively, to remove molecular unwanted species, the filter material may provide a chemical filter medium for removing unwanted species by sorption, for example by adsorption or ion exchange, whereby the filter material binds with the molecular unwanted species to retain them within the filter. The filter material has a plurality of passageways, for example pores (i.e., interconnected hollow voids), extending therethrough to allow fluid flow through the filter.

The filter may be a modular filter including first and second sub-filters arranged to allow fluid flow therethrough in series for progressively removing different unwanted species from the therapeutic agent, for example unwanted species of varying sizes and/or varying molecular composition. Accordingly, the first sub-filter may include a filter material different to that of the second sub-filter. The filter may similarly include a third, a fourth and so on sub-filters.

Suitable filter materials include a foam made of a cellulose, a polyurethane, a polyester, a polyether, a collagen or the like. While a foam is described herein, it would be apparent other structures, such as a membrane or a sheet or similar may be used in place of a foam and the properties described in relation to foams apply equally to membranes or sheets. The foam includes a plurality of passageways in the form of interconnected pores extending therethrough to allow fluid flow through the filter. The foam may be any foam capable of removing particulate unwanted species from an insulin solution. The foam may remove particulate unwanted species from an insulin solution by a size exclusion process.

Particularly suitable filter materials are disclosed in earlier patent applications, including U.S. Pat. Nos. 4,083,906 and 11,197,949, the contents of which are incorporated herein by reference.

Suitable filter materials include an ion-exchange resin, including functionalised porous or gel polymers, which may remove unwanted species from an insulin solution by a gel permeation chromatography process. Moreover, gel polymers may be used to coat passageways in the filter material.

Generally, the filter material may be selected to have at least one material property that may facilitate the infusion of insulin at a single infusion site over an extended period of time, and thereby increase wear times, for example at least four days, including four to seven days, seven or more days, seven to 10 days, 10 or more days, and 14 or more days.

Advantageously, where the filter 205 is disposed within the tubing connector 200, the filter 205 is not constrained (e.g., in size) by the size of the body 35a of the infusion hub 35, and thus body 35a of the infusion hub 35 can be made smaller without compromising the functionality of the filter 205. This allows for a greater range of filters and/or filter arrangements to be used (e.g., varying in size, in number of filters in a particular arrangement, etc.).

Advantageously, where the filter 205 is disposed within the tubing connector 200, the tubing connector 200 can be retrofitted to infusion sets 1000 (e.g., as shown in FIGS. 1A and 1 B) by replacing the tubing connector 35b, to provide the benefits of the presently described infusion set 2000 without having to replace all parts of a patient's existing infusion set 1000, thus reducing wastage.

Advantageously, where the filter 205 is disposed within the tubing connector 200, the tubing connector 200, and therefore the presently described infusion 2000, does not increase the component piece count over the infusion set 1000, e.g., as shown in FIGS. 1A and 1 B.

Advantageously, where the filter 305 is disposed within the cavity 320 of the fluid transfer part 300, the overall dimensions of the infusion hub 35, and the visual appearance of the infusion set 3000, does not change over the infusion set 1000. Further, the presently described infusion set 3000 does not increase the component piece count over infusion set 1000. As such, the patient can receive the benefits of the filter 305 of the infusion set 3000, whilst the infusion set 3000 remains portable for the patient.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, or groups described in conjunction with a particular aspect, embodiment or example of the invention are to be understood to be applicable to any other aspect, embodiment or example described herein unless incompatible therewith. All the features disclosed in this specification (including any accompanying claims, abstract and drawings), and/or all the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

1. An infusion hub for subcutaneous infusion of a therapeutic agent into a patient, the infusion hub comprising:
a casing;
a cannula for insertion into a patient;
a fluid transfer part connected to the casing and in fluid communication with the cannula; and,
a tubing connector configured to engage the fluid transfer part and connectable to tubing for receiving a therapeutic agent from a pump;
wherein the cannula defines a downstream end of a fluid flow path extending between a pump and the patient via the fluid transfer part; and,
wherein the infusion hub comprises a filter disposed in the tubing connector.

2. An infusion hub according to claim 1, wherein an interface of the fluid transfer part comprises a sealing membrane configured to prevent egress of fluid out of the fluid transfer part from the fluid flow path.

3. An infusion hub according to claim 2, wherein when the tubing connector is secured to the fluid transfer part, a needle of the tubing connector pierces the sealing membrane of the fluid transfer part.

4. An infusion hub according to any of claims 1 to 3, wherein the tubing connector comprises an upstream fluid lumen, a downstream fluid lumen, and a cavity provided between the upstream fluid lumen and the downstream fluid lumen;
wherein the filter is disposed in the fluid flow path within the cavity.

5. An infusion hub according to any of claims 1 to 4, wherein the filter provides a physical filter medium for removing unwanted species by size exclusion.

6. An infusion hub according to any of claims 1 to 5, wherein the filter provides a chemical filter medium for removing unwanted species by sorption.

7. An infusion hub according to any of claims 1 to 6, wherein the filter is a modular filter comprising first and second sub-filters arranged to allow fluid flow therethrough in series.

8. An infusion hub according to claim 7, wherein the first sub-filter comprises a filter material and the second sub-filter comprises a further filter material different to the filter material.

9. An infusion hub according to any of claims 1 to 8, wherein the filter comprises a foam or a sheet or a membrane comprising: a cellulose, a polyurethane (Pll), a polyester, a polyether and/or a collagen material.

10. An infusion set comprising:
an infusion hub according to any of claims 1 to 9; and,
tubing connectable between the tubing connector of the infusion hub and a pump.

11. An infusion set according to claim 10, further comprising a pump for containing and delivering the therapeutic agent to the infusion hub via the tubing.

12. A method of treating a patient via subcutaneous infusion with a therapeutic agent, the method comprising the steps of:
placing an infusion hub according to any of claims 1 to 9 at an infusion site of a patient,
connecting, via tubing, a pump fluidly connected to a source of a therapeutic agent to the tubing connector,
priming the tubing with the therapeutic agent, and
injecting the patient with the therapeutic agent.

13. A method of treating a patient via subcutaneous infusion with a therapeutic agent, the method comprising the steps of:
providing an infusion hub according to any of claims 1 to 9,
connecting, via tubing, a pump fluidly connected to a source of a therapeutic agent to the tubing connector,
priming the tubing with the therapeutic agent,
placing the infusion hub at an infusion site of a patient, and
injecting the patient with the therapeutic agent.

14. An infusion hub for subcutaneous infusion of a therapeutic agent into a patient, the infusion hub comprising:
a casing;
a cannula for insertion into a patient;
a fluid transfer part, connected to the casing and in fluid communication with the cannula; and,
a tubing connector configured to engage the fluid transfer part and connectable to tubing for receiving a therapeutic agent from a pump;
wherein the cannula defines a downstream end of a fluid flow path extending between a pump and the patient via the fluid transfer part; and,
wherein the infusion hub comprises a filter disposed in the fluid flow path within the fluid transfer part.

15. An infusion hub according to claim 14, wherein the filter is disposed in the fluid flow path between an interface of the fluid transfer part and the cannula.

16. An infusion set comprising:
an infusion hub according to claim 14 or 15; and,
tubing connectable between the tubing connector of the infusion hub and a pump.

17. An infusion set according to claim 16, further comprising a pump for containing and delivering the therapeutic agent to the infusion hub via the tubing.
